(19) United States
(12) Patent Application Publication
(10) Pub. No.: US 2023/0368385 A1
(43) Pub. Date: Nov. 16, 2023

(54) SYMPTOM RECORDING DEVICE, SYMPTOM RECORDING METHOD, AND PROGRAM

(71) Applicant: Terumo Kabushiki Kaisha, Tokyo (JP)

(72) Inventors: Yoshihito MACHIDA, Ashigarakami-gun (JP); Xiaowei LU, Ashigarakami-gun Kanagawa (JP)

(73) Assignee: TERUMO KABUSHIKI KAISHA, Tokyo (JP)

(21) Appl. No.: 18/360,965

(22) Filed: Jul. 28, 2023

Related U.S. Application Data
(63) Continuation of application No. PCT/JP2022/001849, filed on Jan. 19, 2022.

(30) Foreign Application Priority Data
Jan. 29, 2021 (JP) 2021-013718

(51) Int. Cl. G06T 7/00; H04N 23/63; G06T 7/70

(52) U.S. Cl. CPC G06T 7/0012; H04N 23/631; H04N 23/633; G06T 7/70; G06T 2207/30101

(57) ABSTRACT

A symptom recording device includes a control unit that before a neck image is captured, confirms a state of a jugular vein, to the control unit displays an image obtained by an imaging element to be used for the captured neck image, the image to be used a preview image, outputs range instruction data for giving an instruction of an imaging range including a neck, thereby supporting the captured neck image, and records the captured neck image.

### CROSS-REFERENCES TO RELATED APPLICATIONS

This application is a continuation of International Application No. PCT/JP2022/001849 filed on Jan. 19, 2022, which claims priority to Japanese Application No. 2021-013718 filed on Jan. 29, 2021, the entire content of both of which is incorporated herein by reference.

### TECHNOLOGICAL FIELD

The present disclosure relates to a symptom recording device, a symptom recording method, and a non-transitory computer-readable medium storing a computer program.

### BACKGROUND DISCUSSION

Rise in a central venous pressure manifests as jugular venous distension. Methods for estimating circulatory conditions such as cardiac function by estimating a central venous pressure have been used clinically.

Japanese Patent Application Publication No. 2018-514238 A discloses a technique of shooting a video of a right side of the neck in order to determine a central venous pressure.

A method by which intravascular congestion due to heart failure exacerbation determined from appearance can be used to confirm jugular vein distension, but experience and knowledge are required. Congestion in this particular case refers to an increase in volume of the jugular vein of the neck. In a case where jugular vein distension is to be confirmed visually or by image analysis in telemedicine or telemonitoring, it is necessary to adjust conditions for correctly capturing an image for confirming jugular vein distension, such as positioning. However, it can be difficult for a user having no experience or knowledge to set such conditions.

A medical professional can be a user, and thus, it can be difficult for a user (i.e., the medical professional) having relatively no experience or knowledge to capture an image with which jugular vein distension can be confirmed.

### SUMMARY

The present disclosure makes it rather easy for a user having little or no experience or knowledge to capture an image with which a state of a jugular vein can be confirmed.

A symptom recording device according to an aspect of the present disclosure includes a control unit that, before a neck image is captured can confirm a state of a jugular vein, performs control to display an image obtained by an imaging element, the image to be used for the image capture as a preview image, performs control to output range instruction data for giving an instruction of an imaging range including a neck, thereby supporting or assisting with the capturing of the neck image, and recording the captured neck image.

As an embodiment, the range instruction data is data for giving an instruction of a range from under the ear to the clavicle as the imaging range.

As an embodiment, the control unit performs control to output posture instruction data for giving an instruction of a posture before the neck image is captured.

As an embodiment, the control unit performs control to output clothing instruction data for giving an instruction of clothes or hairstyle instruction data for giving an instruction of a hairstyle before the neck image is captured.

As an embodiment, the control unit performs control to output condition instruction data for giving an instruction of imaging conditions including ambient brightness before the neck image is captured.

As an embodiment, the control unit sets imaging conditions including sensitivity of the imaging element before the neck image is captured.

As an embodiment, the control unit further records date and time data indicating date and time when the neck image is captured.

As an embodiment, the control unit further records angle data indicating an angle at which the neck image is captured.

As an embodiment, the control unit determines whether the imaging range is included in the image obtained by the imaging element and performs control to display a figure representing the imaging range so as to be superimposed on the preview image in a case where it is determined that the imaging range is included.

As an embodiment, the control unit determines whether the imaging range is included in the image obtained by the imaging element and records the image obtained by the imaging element as the neck image in a case where it is determined that the imaging range is included.

As an embodiment, the control unit performs control to display the captured neck image and performs control to output question data for asking whether the imaging range is included in the captured neck image and supports re-image capture of the neck image in a case where an answer that the imaging range is not included is input into the control unit.

As an embodiment, the control unit performs control to display a previously recorded neck image together with the captured neck image.

As an embodiment, the control unit analyzes the captured neck image, determines whether the imaging range is included in the captured neck image and supports re-image capture of the neck image in a case where it is determined that the imaging range is not included.

As an embodiment, the symptom recording device further includes a communication unit that is controlled by the control unit and transmits the neck image to a server.

As an embodiment, the control unit supports re-image capture of the neck image in a case where indication data indicating that the transmitted neck image does not include the imaging range is transmitted from the server and received by the communication unit.

A symptom recording method according to an aspect of the present disclosure includes, before a neck image is captured, confirms a state of a jugular vein, the method including displaying an image obtained by an imaging element, the image to be used for the captured neck image as a preview image; outputting range instruction data for giving an instruction of an imaging range including the neck, thereby supporting or assisting with the capturing of neck image by a control unit, and recording the captured neck image by the control unit.

A non-transitory computer-readable medium storing a computer program according to an aspect of the present disclosure causes a computer to execute a process, before capturing a neck image, confirms a state of a jugular vein, the process including displaying an image obtained by an imaging element, the image to be used for the captured neck image as a preview image, outputting range instruction data for giving an instruction of an imaging range including the neck, thereby supporting or assisting with the capturing of the neck image, and recording the captured neck image.

According to the present disclosure, it becomes relatively easy for a user having little or no experience or knowledge to capture an image with which a state of a jugular vein can be confirmed.

### BRIEF DESCRIPTION OF THE DRAWINGS

FIG. 1 is a view illustrating a configuration of a system according to an embodiment of the present disclosure.

FIG. 2 is a block diagram illustrating a configuration of a symptom recording device according to an embodiment of the present disclosure.

FIG. 3 is a view illustrating a screen example of the symptom recording device according to the embodiment of the present disclosure.

FIG. 4 is a view illustrating a screen example of the symptom recording device according to the embodiment of the present disclosure.

FIG. 5 is a view illustrating a screen example of the symptom recording device according to the embodiment of the present disclosure.

FIG. 6 is a view illustrating a screen example of the symptom recording device according to the embodiment of the present disclosure.

FIG. 7 is a flowchart illustrating operation of the symptom recording device according to the embodiment of the present disclosure.

### DETAILED DESCRIPTION

Set forth below with reference to the accompanying drawings is a detailed description of embodiments of a symptom recording device, a symptom recording method, and a non-transitory computer-readable medium storing a computer program.

In the drawings, the same or corresponding portions are denoted by the same reference numerals. In the description of the present embodiment, description of the same or corresponding parts will be omitted or simplified as appropriate.

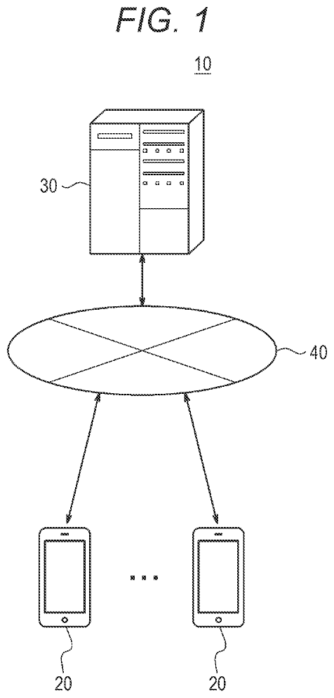

A configuration of a system 10 according to the present embodiment will be described with reference to FIG. 1.

The system 10 according to the present embodiment includes a plurality of symptom recording devices 20 and at least one server 30.

Each of the plurality of symptom recording devices 20 is used by a user such as a patient, a family member of the patient, a caregiver, or a medical worker. The patient can be, for example, a patient with heart failure.

The number of symptom recording devices 20 is not limited to a plurality, and may be only one. Hereinafter, one symptom recording device 20 will be described for convenience of description.

The symptom recording device 20 can be, for example, held by a user.

Alternatively, the symptom recording device 20 is installed at the patient's home. The symptom recording device 20 can be, for example, a general-purpose terminal such as a mobile phone, a smartphone, a tablet, or a personal computer (PC), or a dedicated terminal such as a gadget.

The symptom recording device 20 is communicable with the server 30 via a network 40.

The server 30 can be installed, for example, in a facility such as a data center. The server 30 can be, for example, a server computer belonging to a cloud computing system or other computing systems.

The network 40 includes the Internet, at least one wide area network (WAN), at least one metropolitan area network (MAN), or any combination of at least one wide area network (WAN) and at least one metropolitan area network (MAN). The network 40 may include at least one wireless network, at least one optical network, or any combination of at least one wireless network and at least one optical network. The wireless network can be, for example, an ad hoc network, a cellular network, a wireless local area network (LAN), a satellite communication network, or a terrestrial microwave network.

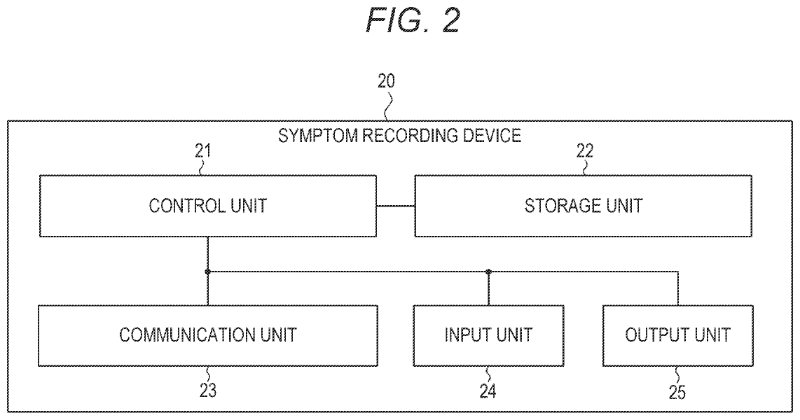

Outline of the present embodiment will be described with reference to FIGS. 2, 4 and 6.

In the symptom recording device 20 according to the present embodiment, before a neck image 56 for confirming a state of a jugular vein is captured, a control unit 21 performs control to display an image obtained by the imaging element to be used for the image capture as a preview image 52 and performs control to output range instruction data D1 for giving an instruction of an imaging range including the neck, thereby supporting (or assisting) with the capturing of the neck image. The control unit 21 records the captured neck image 56.

In the present embodiment, when the user views the preview image 52, the user can receive an instruction on the imaging range for obtaining an image with which the state of the jugular vein can be confirmed. Thus, according to the present embodiment, it becomes relatively easy for a user having little or no experience or knowledge to capture an image with which the state of the jugular vein can be confirmed.

The imaging range can include at least a region of the neck where the state of the jugular vein can be confirmed, and in the present embodiment, can include a range from under the ear to the clavicle. In other words, the range instruction data D1 is data for giving an instruction of a range from under the ear to the clavicle as the imaging range. According to the present embodiment, as a result of the imaging range including a range up to the clavicle, it is possible to increase estimated accuracy of the central venous pressure by height estimation. As a modification of the present embodiment, the imaging range may include a range up to the sternal angle. Also, the higher the central venous pressure, the higher a position of the pulsation (i.e., pulse of the jugular vein in the neck), and in some patients the pulsation may appear just below the ear. According to the present embodiment, as a result of the imaging range including a range to under the ear, it is possible to reduce image capture errors for a patient having a relatively high central venous pressure. In other words, it becomes relatively easy to avoid a situation in which the position of the pulsation of the patient having a high central venous pressure is not included in the imaging range. As a modification of the present embodiment, the imaging range may include a range to the earlobe.

A configuration of the symptom recording device 20 according to the present embodiment will be described with reference to FIG. 2.

The symptom recording device 20 can include the control unit 21, a storage unit 22, a communication unit 23, an input unit 24, and an output unit 25.

The control unit 21 can include at least one processor, at least one programmable circuit, at least one dedicated circuit, or any combination of at least one processor, at least one programmable circuit, and at least one dedicated circuit. The processor can be a general-purpose processor such as a central processing unit (CPU) or a graphics processing unit (GPU), or a dedicated processor specialized for specific processing. The programmable circuit can be, for example, a field-programmable gate array (FPGA). The dedicated circuit can be, for example, an application specific integrated circuit (ASIC). The control unit 21 executes processing related to operation of the symptom recording device 20 while controlling each unit of the symptom recording device 20.

The storage unit 22 can include at least one semiconductor memory, at least one magnetic memory, at least one optical memory, or any combination of the at least one semiconductor memory, the at least one magnetic memory, and the at least one optical memory. The semiconductor memory can be, for example, a random access memory (RAM) or a read only memory (ROM). The RAM can be, for example, a static random access memory (SRAM) or a dynamic random access memory (DRAM). The ROM can be, for example, an electrically erasable programmable read only memory (EEPROM). The storage unit 22 can function as, for example, a main storage, an auxiliary storage, or a cache memory. The storage unit 22 stores data to be used for the operation of the symptom recording device 20 and data obtained by the operation of the symptom recording device 20.

The communication unit 23 includes at least one communication interface. The communication interface can be, for example, an interface compatible with mobile communication standards such as long term evolution (LTE), the 4th generation (4G) standards, or the 5th generation (5G) standards, an interface compatible with short-range wireless communication standards such as Bluetooth®, or a LAN interface. The communication unit 23 receives data to be used for the operation of the symptom recording device 20 and transmits data obtained by the operation of the symptom recording device 20.

The input unit 24 can include at least two input interfaces. One input interface is imaging equipment including an imaging element such as a camera. The other input interface can be, for example, a physical key, a capacitance key, a pointing device, a touch screen provided integrally with a display, or a microphone. The input unit 24 receives operation of inputting data to be used for the operation of the symptom recording device 20. The input unit 24 may be connected to the symptom recording device 20 as external input equipment instead of being provided in the symptom recording device 20. As a connection interface, for example, an interface compatible with standards such as universal serial bus (USB), a high-definition multimedia interface (HDMI®), or Bluetooth can be used.

The output unit 25 can include at least two output interfaces. One output interface is a display such as a liquid crystal display (LCD) or an organic electro luminescence (EL) display. The other output interface can be, for example, a speaker. The output unit 25 outputs data obtained by the operation of the symptom recording device 20. The output unit 25 may be connected to the symptom recording device 20 as external output equipment instead of being provided in the symptom recording device 20. As a connection interface, for example, an interface compatible with standards such as USB, HDMI, or Bluetooth can be used.

Functions of the symptom recording device 20 are implemented by a program according to the present embodiment being executed by a processor as the control unit 21. In other words, the functions of the symptom recording device 20 are implemented by software. The program causes a computer to function as the symptom recording device 20 by causing the computer to execute the operation of the symptom recording device 20. In other words, the computer functions as the symptom recording device 20 by executing the operation of the symptom recording device 20 according to the program.

The program can be stored in a non-transitory computer-readable medium. The non-transitory computer-readable medium can be, for example, a flash memory, a magnetic recording device, an optical disc, a magneto-optical recording medium, or a ROM. The program can be distributed, for example, by selling, transferring, or lending a portable medium, such as secure digital (SD) card, a digital versatile disc (DVD), or a compact disc read only memory (CD-ROM), storing the program. The program may be distributed by storing the program in a storage of a server and transferring the program from the server to another computer. The program may be provided as a program product.

The computer temporarily stores, for example, a program stored in a portable medium or a program transferred from the server in the main storage. Then, the computer reads the program stored in the main storage by the processor and executes processing according to the read program by the processor. The computer may read the program directly from the portable medium and execute the processing according to the program. Each time the program is transferred from a server to a computer, the computer may sequentially execute processing according to the received program. The processing may be executed by what is called an application service provider (ASP) service in which the functions are implemented only by execution instructions and result acquisition instead of the program being transferred from the server to the computer. The program includes information that is provided for processing by an electronic computer and is equivalent to the program. For example, data that is not a direct command to the computer but has property that defines processing of the computer corresponds to the “information equivalent to the program”.

Some or all of the functions of the symptom recording device 20 may be implemented by a programmable circuit or a dedicated circuit as the control unit 21. In other words, some or all of the functions of the symptom recording device 20 may be implemented by hardware.

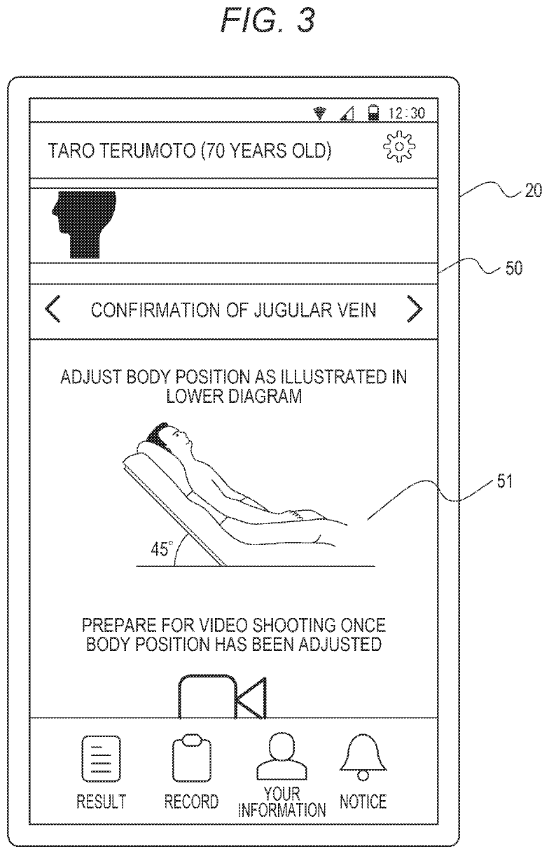

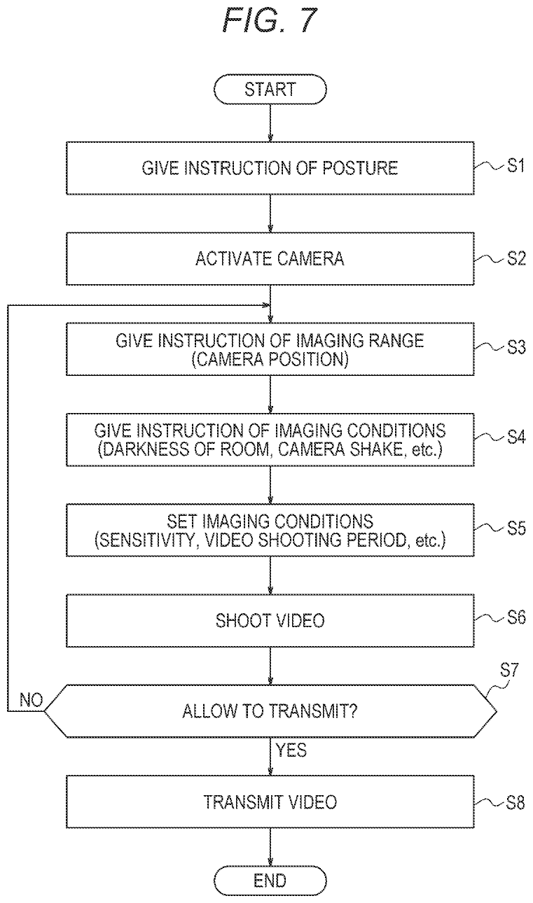

The operation of the symptom recording device 20 according to the present embodiment will be described with reference to FIG. 7 using the example of a screen 50 of the symptom recording device 20 illustrated in FIGS. 3 to 6. This operation corresponds to a symptom recording method according to the present embodiment.

In S1, the control unit 21 performs control to output posture instruction data D2 for giving an instruction of a posture to a user. Display as the output unit 25 is controlled by the control unit 21 to display the posture instruction data D2 on the screen 50. Alternatively, a speaker as the output unit 25 is controlled by the control unit 21 and outputs the posture instruction data D2 by speech.

In the present embodiment, as illustrated in FIG. 3, the control unit 21 causes a display to display an illustration 51 for instructing the user of an angle with respect to a supine position as the posture instruction data D2. The angle indicated by the illustration 51 can be, for example, 30° to 45° and the angle is 45° in the example of FIG. 3. In other words, in the example of FIG. 3, the user is instructed to adjust the posture of the patient to be in a state of rising from the supine position by 45°. In the example of FIG. 3, the control unit 21 further causes the display to display messages, for example, such as “Adjust the body position as illustrated in the lower diagram” and “Prepare for video shooting once the body position has been adjusted”.

As a modification of the present embodiment, the control unit 21 may support image capture of a posture image such as an image of the entire patient and may record the captured posture image. Specifically, the control unit 21 may cause the storage unit 22 to store the image of the posture obtained by an imaging element of imaging equipment as the input unit 24. According to this modification, it becomes relatively easy to ensure that the patient is correctly positioned. As a result, estimation accuracy of the central venous pressure can be improved.

As a further modification, the control unit 21 may cause the display to display an image of the previously recorded posture together with the captured image of the posture. According to this modification, it becomes relatively easy to confirm whether the captured image by the patient is in the correct posture. [0063] As a modification of the present embodiment, it may be possible to select a sitting position or a semi-sitting position by mode selection. According to this modification, information indicating which mode has been selected can be used as additional information for diagnosis.

As a modification of the present embodiment, the control unit 21 may further perform control to output clothing instruction data D3a for giving an instruction of clothes. The display may be controlled by the control unit 21 to display the clothing instruction data D3a on the screen 50. Alternatively, the speaker may be controlled by the control unit 21 to output the clothing instruction data D3a by speech. For example, as the clothing instruction data D3a, the control unit 21 may cause the display to display a clothing instruction message such as “Please wear clothing that allows you to see from under the ear to the clavicle” or may cause a speaker to output the clothing instruction message by speech.

As a modification of the present embodiment, the control unit 21 may further perform control to output hairstyle instruction data D3b for giving an instruction of a hairstyle. The display may be controlled by the control unit 21 to display the hairstyle instruction data D3b on the screen 50. Alternatively, the speaker may be controlled by the control unit 21 to output the hairstyle instruction data D3b by speech. For example, the control unit 21 may cause a hairstyle instruction message such as “If you have long hair, please put your hair together” to be displayed on the display as the hairstyle instruction data D3b or may cause a speaker to output the hairstyle instruction message by speech.

In S2, the control unit 21 activates the imaging equipment as the input unit 24. Specifically, the control unit 21, for example, activates the camera.

In S3, the control unit 21 performs control to display the image obtained by the imaging element as a preview image 52 and performs control to output the range instruction data D1 for giving an instruction of the imaging range including the neck. The display as the output unit 25 is controlled by the control unit 21 and displays the range instruction data D1 on the screen 50 together with the preview image 52. Alternatively, when the preview image 52 is displayed on the screen 50, the speaker as the output unit 25 is controlled by the control unit 21 to output the range instruction data D1 by speech.

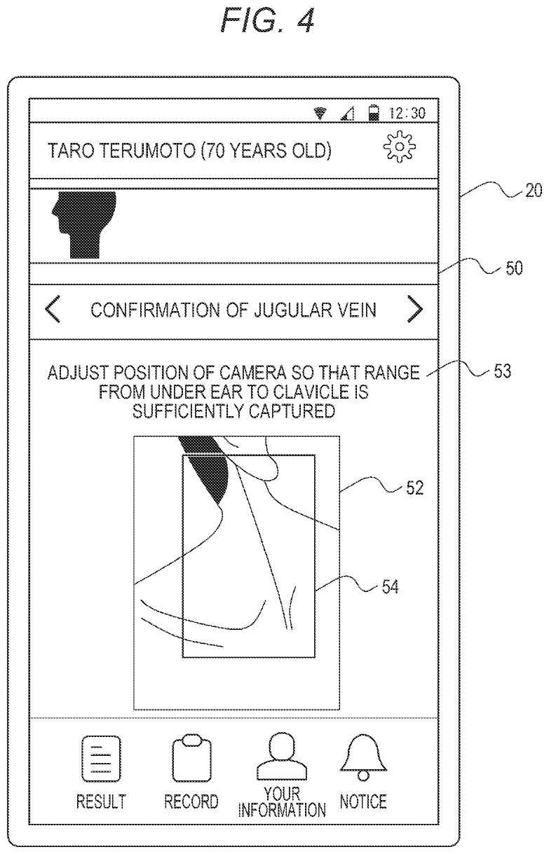

In the present embodiment, as illustrated in FIG. 4, the control unit 21 causes the display to display a range instruction message 53 such as “Adjust the position of the camera so that a range from under the ear to the clavicle is sufficiently captured” as the range instruction data D1 together with the preview image 52. In the example of FIG. 4, the user is instructed to adjust the position of the camera to capture a range from under the ear to the clavicle. The range instruction message 53 may include other messages, for example, such as a message giving an instruction to adjust orientation of the patient's face to turn sideways.

In the present embodiment, the control unit 21 determines whether the image obtained by the imaging element includes the imaging range instructed by the range instruction data D1. As illustrated in FIG. 4, in a case where it is determined that the image obtained by the imaging element includes the imaging range instructed by the range instruction data D1, the control unit 21 performs control to display a FIG. 54 representing the imaging range so as to be superimposed on the preview image 52. The display is controlled by the control unit 21 to display the FIG. 54 so as to be superimposed on the preview image 52. The FIG. 54 may have any shape, color, and pattern, but is a rectangular red frame in the example of FIG. 4. In a case where it is determined that the imaging range instructed by the range instruction data D1 is not included in the image obtained by the imaging element, the control unit 21 performs control to hide the FIG. 54. The display is controlled by the control unit 21 to hide the FIG. 54.

In S4, the control unit 21 performs control to output condition instruction data D4 for giving an instruction of imaging conditions including ambient brightness. The display as the output unit 25 is controlled by the control unit 21 to display the condition instruction data D4 on the screen 50. Alternatively, the speaker as the output unit 25 is controlled by the control unit 21 to output the condition instruction data D4 by speech.

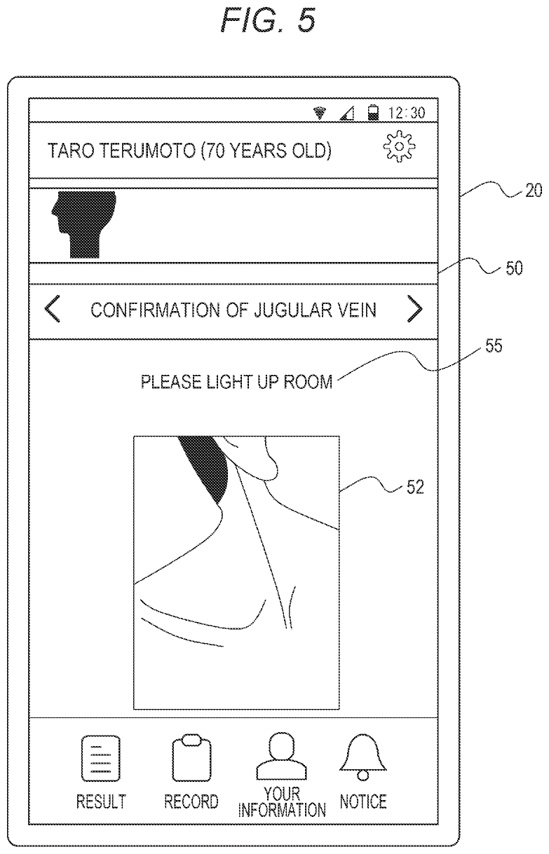

In the present embodiment, as illustrated in FIG. 5, the control unit 21 causes the display to display a condition instruction message 55 such as “Please light up the room” as the condition instruction data D4. In other words, in the example of FIG. 5, the user is instructed to adjust, for example, darkness or brightness of a room to make it sufficiently bright. The condition instruction message 55 may include other messages such as a message giving an instruction to help prevent camera shake of the user or a message, for example, giving an instruction not to include a foreign object.

In S5, the control unit 21 sets imaging conditions including sensitivity of the imaging element. Specifically, the control unit 21 adjusts sensitivity in the background. In the present embodiment, a video is shot as the neck image 56 for confirming the state of the jugular vein, and thus, the control unit 21 may set a shooting period as part of the imaging conditions. The control unit 21 may set other conditions as part of the imaging conditions.

The control unit 21 executes processing from S3 to S5 to support the image capture of the neck image 56.

In S6, the control unit 21 records the captured neck image 56. Specifically, for example, the control unit 21 stores the captured neck image 56 in the storage unit 22.

In the present embodiment, the control unit 21 causes the storage unit 22 to store, as the neck image 56, a video including, as frames, images obtained by the imaging element for a fixed period after S5. The video shooting may be started automatically or may be started in response to user operation such as a shooting button, for example, being tapped via a touch screen as the input unit 24. The video shooting may be terminated automatically or may be terminated in response to user operation, such as a stop button being tapped through the touch screen.

As a modification of the present embodiment, the control unit 21 may analyze an image obtained by the imaging element and determine whether the image includes the imaging range instructed by the range instruction data D1. As a method of image analysis, known methods can be used. Machine learning such as deep learning may be used. In a case where it is determined that the image obtained by the imaging element includes the imaging range instructed by the range instruction data D1, the control unit 21 may record the image as the neck image 56. Specifically, the control unit 21 may cause the storage unit 22 to store, as the neck image 56, a video including, as frames, images obtained by the imaging element and including the imaging range instructed by the range instruction data D1.

As a modification of the present embodiment, the control unit 21 may further record date and time data D5 indicating date and time when the neck image 56 is captured. Specifically, the control unit 21 may store the date and time data D5 in the storage unit 22 together with the captured neck image 56. According to this modification, daily variation of the central venous pressure can be analyzed. Alternatively, whether the neck image 56 is captured in a warm season or a cold season can be known.

As a modification of the present embodiment, the control unit 21 may further record angle data D6 indicating an angle at which the neck image 56 is captured. Specifically, the control unit 21 may store the angle data D6 in the storage unit 22 together with the captured neck image 56. According to this modification, the posture can be estimated from the angle of the camera and the angle of the neck.

In S7, the control unit 21 performs control to display the captured neck image 56 and performs control to output question data D7 for asking whether to transmit the captured neck image 56 to the server 30. The display as the output unit 25 is controlled by the control unit 21 to display the question data D7 on the screen 50 together with the neck image 56. Alternatively, the speaker as the output unit 25 is controlled by the control unit 21 to output the question data D7 by speech when the neck image 56 is displayed on the screen 50.

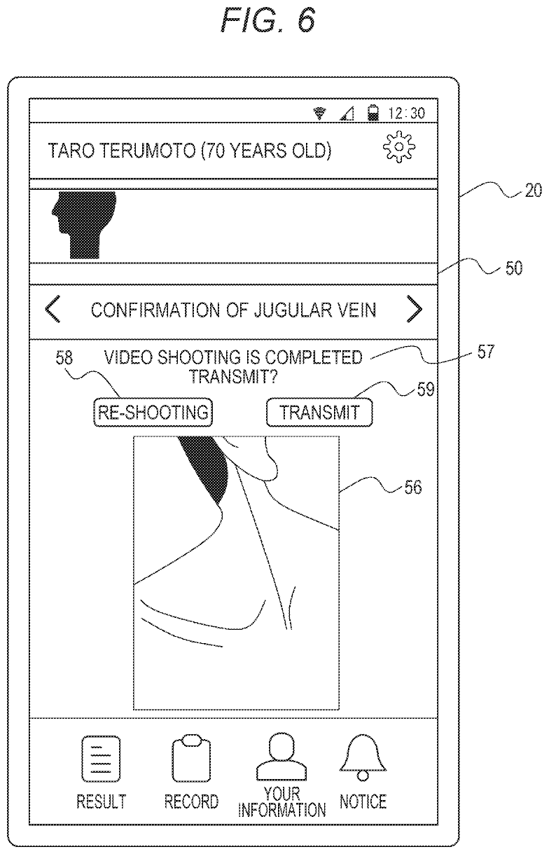

In the present embodiment, as illustrated in FIG. 6, the control unit 21 causes the display to display a confirmation message 57 such as “Video shooting is completed” and “Transmit?” as the question data D7 together with the neck image 56. In the example of FIG. 6, the user is asked whether to transmit the shot video to the server 30.

If an answer that the captured neck image 56 is not transmitted to the server 30 is input via the input unit 24, the processing in and after S3 is executed again. In other words, the control unit 21 supports re-image capture (recapture) of the neck image 56. In a case where an answer that the captured neck image 56 is transmitted to the server 30 is input via the input unit 24, the processing of S8 is executed.

In the present embodiment, as illustrated in FIG. 6, the control unit 21 causes the display to display a re-shooting button 58 with a label “re-shooting” and a transmission button 59 with a label “transmit”. In a case where the re-shooting button 58 is tapped, for example, via the touch screen as the input unit 24, the processing of S3 and subsequent processing are executed again. In a case where the transmission button 59 is tapped via the touch screen, the processing of S8 is executed.

As a modification of the present embodiment, the question data D7 may be data for asking whether the captured neck image 56 includes the imaging range instructed by the range instruction data D1. For example, a confirmation message 57 may be a message such as “Is the range from under the ear to the clavicle shown in the video?”. The user may be asked whether the range from under the ear to the clavicle is shown in the captured video. The labels of the re-shooting button 58 and the transmission button 59 may be, for example, “No” and “Yes”, respectively. Also in this modification, in a case where the re-shooting button 58 is tapped via the touch screen, the processing in and after S3 is executed again. In a case where the transmission button 59 is tapped via the touch screen, the processing of S8 is executed. In a case where an answer that the imaging range instructed by the range instruction data D1 is not included in the captured neck image 56 is input via the input unit 24, the processing of S3 and subsequent processing are executed again. In a case where an answer that the imaging range instructed by the range instruction data D1 is included in the captured neck image 56 is input via the input unit 24, the processing of S8 is executed.

As a modification of the present embodiment, the control unit 21 may display a previously recorded neck image 56 on the display together with the captured neck image 56. According to this modification, it is relatively easy to confirm whether the neck image 56 has been correctly captured.

As a modification of the present embodiment, the control unit 21 may analyze the captured neck image 56 and determine whether the captured neck image 56 includes the imaging range instructed by the range instruction data D1. In other words, whether the range from under the ear to the clavicle is shown in the captured video may be automatically determined instead of being determined by the user. As a method of image analysis, known methods can be used. Machine learning such as deep learning may be used. In this modification, it is not necessary to display the confirmation message 57, the re-shooting button 58, and the transmission button 59. If the control unit 21 determines that the captured neck image 56 does not include the imaging range instructed by the range instruction data D1, the processing of S3 and subsequent processing are executed again. In a case where the control unit 21 determines that the captured neck image 56 includes the imaging range instructed by the range instruction data D1, the processing of S8 is executed.

In S8, the control unit 21 causes the communication unit 23 to transmit the captured neck image 56. The communication unit 23 is controlled by the control unit 21 to transmit the neck image 56 to the server 30.

As one modification of the present embodiment, the server 30 may analyze the captured neck image 56 to determine whether the captured neck image 56 includes the imaging range instructed by the range instruction data D1. In other words, whether the range from under the ear to the clavicle is shown in the captured video may be determined by the server 30. As a method of image analysis, known methods can be used. Machine learning such as deep learning may be used. In a case where the server 30 determines that the captured neck image 56 does not include the imaging range instructed by the range instruction data D1, indication data D8 indicating that the neck image 56 does not include the imaging range may be transmitted from the server 30. In a case where the indication data D8 is transmitted from the server 30 and received by the communication unit 23, the processing of S3 and subsequent processing may be executed again. In other words, the control unit 21 may support re-image capture of the neck image 56. The indication data D8 may include, for example, a comment from a medical institution that has confirmed the neck image 56, such as “the image is dark” or “no jugular vein is shown”.

As described above, in the present embodiment, the symptom recording device 20 gives an instruction of the posture of the subject at the time of measuring the jugular vein. The symptom recording device 20 may indicate the clothes or hairstyle of the subject at the time of measuring the jugular vein. The symptom recording device 20 obtains and displays a video of the vicinity of the neck of the subject. The symptom recording device 20 indicates the imaging range including the vicinity of the neck of the subject in conjunction with the video display. The symptom recording device 20 gives an instruction of or sets imaging conditions that are conditions for shooting a video near the neck of the subject. The symptom recording device 20 records the shot video. Thus, according to the present embodiment, even a user who does not have specialized knowledge can stably shoot a video for confirming the jugular vein distension at home, that is, without failure.

As a modification of the present embodiment, a mode in which the patient captures the neck image 56 by himself/herself may be provided. In this modification, the camera can be, for example, an in-camera. Further, the instruction can be, for example, given by speech. It is not necessary to capture an image of the body position. If the imaging range is captured, image capture automatically starts without button operation. For example, the symptom recording device 20 can be a smartphone. First, messages such as “I will capture an image”, “Please hold the smartphone with the right hand so that the screen faces you”, and “Please press the shooting start button” can be displayed. Next, messages such as “Please move the smartphone a little further away”, “Please move the smartphone a little closer”, “A little more to the right”, or “A little more to the left” are displayed. Next, messages such as “When the face is directed to the left, shooting will start”, “The video is being shot”, and “Shooting is completed” can be sequentially displayed. The body and the smartphone face each other, and thus, explanation will be made with characters. A text or an image may be used to display messages such as “Adjust the position of the smartphone by moving the position away from or close to the position of the smartphone so that the smartphone enters the frame from under the ear to the clavicle”, “Please press the shooting start button when adjustment is completed”, “Shooting will start in 3 seconds”, and “Face left”. Thereafter, messages such as “The video is being shot”, “Shooting is completed”, and “Please confirm the image captured by the smartphone” may be output by speech.

The present disclosure is not limited to the above-described embodiment. For example, a plurality of blocks described in the block diagram may be integrated, or one block may be divided. Instead of executing the plurality of steps described in the flowcharts in chronological order according to the description, the processes or steps may be executed in parallel or in a different order, depending on the processing capability of the device executing the steps or as needed. In addition, modifications can be made within a scope not departing from the gist of the present disclosure.

For example, the processing of S1 may be omitted. Alternatively, the processing of S4, the processing of S5, or both of them may be omitted.

For example, instead of a video being shot, a still image may be captured as the neck image 56. In a case where it is determined whether the neck image 56 includes the imaging range instructed by the range instruction data D1, it may be further determined whether the neck image 56 satisfies the imaging conditions instructed by the condition instruction data D4. Alternatively, instead of whether the neck image 56 includes the imaging range instructed by the range instruction data D1 being determined, it may be determined whether the neck image 56 satisfies the imaging conditions instructed by the condition instruction data D4.

For example, when re-image capture of the neck image 56 is supported, the control unit 21 may notify the user of a reason why re-image capture is necessary, for example, such as an imaging range, illuminance, posture, a foreign object such as a finger of a user, or camera shake.

The detailed description above describes embodiments of a symptom recording device, a symptom recording method, and a non-transitory computer-readable medium storing a computer program. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A symptom recording device, before a neck image is captured, confirms a state of a jugular vein, the recording device comprising:
a control unit configured to:
display an image obtained by an imaging element, the image to be used for the captured neck image as a preview image;
output range instruction data for giving an instruction of an imaging range; and
record the captured neck image.

2. The symptom recording device according to claim 1, wherein the range instruction data is data for giving an instruction of a range from under an ear to a clavicle as the imaging range.

3. The symptom recording device according to claim 1, wherein the control unit is configured to output posture instruction data for giving an instruction of a posture before the neck image is captured.

4. The symptom recording device according to claim 1, wherein the control unit is configured to output clothing instruction data for giving an instruction of clothes or hairstyle instruction data for giving an instruction of a hairstyle before the neck image is captured.

5. The symptom recording device according to claim 1, wherein the control unit is configured to output condition instruction data for giving an instruction of imaging conditions including ambient brightness before the neck image is captured.

6. The symptom recording device according to claim 1, wherein the control unit is configured to set imaging conditions including sensitivity of the imaging element before the neck image is captured.

7. The symptom recording device according to claim 1, wherein the control unit is further configured to record date and time data indicating date and time when the neck image is captured.

8. The symptom recording device according to claim 1, wherein the control unit is further configured to record angle data indicating an angle at which the neck image is captured.

9. The symptom recording device according to claim 1, wherein the control unit is configured to determine whether the imaging range is included in the image obtained by the imaging element and to display a figure representing the imaging range so as to be superimposed on the preview image in a case where it is determined that the imaging range is included.

10. The symptom recording device according to claim 1, wherein the control unit is configured to determine whether the imaging range is included in the image obtained by the imaging element and to record the image obtained by the imaging element as the neck image in a case where it is determined that the imaging range is included in the image obtained by the imaging element.

11. The symptom recording device according to claim 1, wherein the control unit is configured to:
display the captured neck image;
output question data for asking whether the imaging range is included in the captured neck image; and
support re-image capture of the neck image in a case where an answer that the imaging range is not included is input into the control unit.

12. The symptom recording device according to claim 1, wherein the control unit is configured to display a previously recorded neck image together with the captured neck image.

13. The symptom recording device according to claim 1, wherein the control unit is configured to:
analyze the captured neck image;
determine whether the imaging range is included in the captured neck image; and
support re-image capture of the neck image in a case where it is determined that the imaging range is not included in the captured neck image.

14. The symptom recording device according to claim 1, further comprising:
a communication unit configured to transmit the neck image to a server.

15. The symptom recording device according to claim 14, wherein the control unit is configured to support re-image capture of the neck image in a case where indication data indicating that the transmitted neck image does not include the imaging range is transmitted from the server and received by the communication unit.

16. A symptom recording method, before a neck image is captured, confirms a state of a jugular vein, the method comprising:
displaying an image obtained by an imaging element, the image to be used for the captured neck image as a preview image;
outputting, by a control unit, range instruction data for giving an instruction of an imaging range; and
recording, by the control unit, the captured neck image.

17. The method according to claim 16, further comprising:
determining whether the imaging range is included in the image obtained by the imaging element; and
displaying a figure representing the imaging range so as to be superimposed on the preview image in a case where it is determined that the imaging range is included.

18. The method according to claim 16, further comprising:
determining whether the imaging range is included in the image obtained by the imaging element; and
recording the image obtained by the imaging element as the neck image in a case where it is determined that the imaging range is included in the image obtained by the imaging element.

19. The method according to claim 16, further comprising:
displaying the captured neck image;
outputting question data for asking whether the imaging range is included in the captured neck image; and
supporting re-image capture of the neck image in a case where an answer that the imaging range is not included is input into the control unit.

20. A non-transitory computer-readable medium storing a computer program that causes a computer to execute a process before a neck image is captured, confirms a state of a jugular vein, the process comprising:
displaying an image obtained by an imaging element, the image to be used for the captured neck image as a preview image;
outputting range instruction data for giving an instruction of an imaging range; and
recording the captured neck image.
